SEQUENCE LISTING (25 sequences)

SEQ ID NO: 1 - DNA, 21 bp, Artificial Sequence
  Synthetic polynucleotide
cattctatccgctggaagatg

SEQ ID NO: 2 - RNA, 21 nt, Artificial Sequence
  Synthetic polynucleotide
cauucuauccgcuggaagaug

SEQ ID NO: 3 - DNA, 21 bp, Artificial Sequence
  Synthetic polynucleotide
tcttccagcggatagaatggc

SEQ ID NO: 4 - RNA, 21 nt, Artificial Sequence
  Synthetic polynucleotide
ucuuccagcggauagaauggc

SEQ ID NO: 5 - DNA, 21 bp, Artificial Sequence
  Synthetic polynucleotide
acgccaaaaacataaagaaag

SEQ ID NO: 6 - RNA, 21 nt, Artificial Sequence
  Synthetic polynucleotide
acgccaaaaacauaaagaaag

SEQ ID NO: 7 - DNA, 21 bp, Artificial Sequence
  Synthetic polynucleotide
ttctttatgtttttggcgtct

SEQ ID NO: 8 - RNA, 21 nt, Artificial Sequence
  Synthetic polynucleotide
uucuuuauguuuuuggcgucu

SEQ ID NO: 9 - DNA, 21 bp, Artificial Sequence
  Synthetic polynucleotide
ggtaaagttgttttatttttt

SEQ ID NO: 10 - RNA, 21 nt, Artificial Sequence
  Synthetic polynucleotide
gguaaaguuguuuuauuuuuu

SEQ ID NO: 11 - DNA, 21 bp, Artificial Sequence
  Synthetic polynucleotide
aaaatggaacaactttaccga

SEQ ID NO: 12 - RNA, 21 nt, Artificial Sequence
  Synthetic polynucleotide
aaaauggaacaacuuuaccga

SEQ ID NO: 13 - DNA, 21 bp, Artificial Sequence
  Synthetic polynucleotide
cattctatccgcuggaagaug

SEQ ID NO: 14 - DNA, 21 bp, Artificial Sequence
  Synthetic polynucleotide
cauucuauccgctggaagatg

SEQ ID NO: 15 - DNA, 21 bp, Artificial Sequence
  Synthetic polynucleotide
tcttccagcggatagaatggc

SEQ ID NO: 16 - DNA, 21 bp, Artificial Sequence
  Synthetic polynucleotide
ucuuccagcggatagaauggc

SEQ ID NO: 17 - DNA, 21 bp, Artificial Sequence
  Synthetic polynucleotide
tcttccagcggauagaatggc

SEQ ID NO: 18 - DNA, 21 bp, Artificial Sequence
  Synthetic polynucleotide
tctuccagcggauagaauggc

SEQ ID NO: 19 - DNA, 21 bp, Artificial Sequence
  Synthetic polynucleotide
ucutccagcggatagaatggc

SEQ ID NO: 20 - DNA, 21 bp, Artificial Sequence
  Synthetic polynucleotide
tcttccagcggauagaauggc

SEQ ID NO: 21 - DNA, 21 bp, Artificial Sequence
  Synthetic polynucleotide
ucuuccagcggatagaatggc

SEQ ID NO: 22 - DNA, 21 bp, Artificial Sequence
  Synthetic polynucleotide
tcttccagcggatagaatggc

SEQ ID NO: 23 - DNA, 21 bp, Artificial Sequence
  Synthetic polynucleotide
ucuuccagcggauagaauggc

SEQ ID NO: 24 - DNA, 21 bp, Artificial Sequence
  Synthetic polynucleotide
tcttccagcggauagaauggc

SEQ ID NO: 25 - DNA, 21 bp, Artificial Sequence
  Synthetic polynucleotide
ucuuccagcggatagaatggc

(19) United States
(12) Patent Application Publication
(10) Pub. No.: US 2011/0289607 A1
(43) Pub. Date: Nov. 24, 2011

(54) METHOD OF INHIBITING GENE EXPRESSION

(75) Inventors: Kumiko TEI, Tokyo (JP); Takahide Kaji, Yokohama-shi (JP); Ryu Ueda, Tokyo (JP); Kaoru Saigo, Tokyo (JP)

(73) Assignees: Kaoru SAIGO, Tokyo (JP); ALPHAGEN CO., LTD., Kanagawa (JP)

(21) Appl. No.: 13/176,536

(22) Filed: Jul. 5, 2011

Related U.S. Application Data
(63) Continuation of application No. 10/849,912, filed on May 21, 2004, now abandoned.
Application No. 10/849,912 is a continuation of application No. PCT/JP02/12183, filed on Nov. 21, 2002.

(30) Foreign Application Priority Data
Nov. 21, 2001 (JP) 2001-355896

(51) Int. Cl. A01K 67/00; C12N 5/07; C12N 5/0787; C12N 5/0786; C12N 5/0793; C12N 5/077; C12N 5/073; C12N 5/078; C12N 7/00; C12N 1/14; C12N 1/10; C12N 1/20; C12N 1/16; A61K 31/713; C12Q 1/70; C12Q 1/02; A01H 5/00; C12Q 1/68; C07H 21/00; C12N 5/071

(52) U.S. Cl. 800/8; 435/375; 435/367; 435/369; 435/348; 435/358; 435/235.1; 435/254.1; 435/258.1; 435/252.1; 435/255.1; 514/44 A; 435/5; 435/29; 800/298; 435/6.1; 435/6.18; 536/24.5; 536/23.1

(57) ABSTRACT

The present invention relates to a method for inhibiting expression of a target gene, which comprises introducing a cell, tissue, or individual organism with a double-stranded polynucleotide comprising DNA and RNA having a substantially identical nucleotide sequence with at least a partial nucleotide sequence of the target gene.

### CROSS-REFERENCE TO RELATED APPLICATIONS

The present application is a continuation of U.S. application Ser. No. 10/849,912, filed May 21, 2004, which is a continuation of PCT/JP02/12183, filed Nov. 21, 2002, which claims priority to JP 2001-355896, filed Nov. 21, 2001, the entire disclosures of which are hereby incorporated by reference.

### BACKGROUND OF THE INVENTION

1. Field of the Invention

The present invention relates to a method for inhibiting expression of a target gene by introducing a double-stranded polynucleotide comprising DNA and RNA having a substantially identical nucleotide sequence with at least a partial nucleotide sequence of the target gene into a cell, tissue, or individual organism.

2. Discussion of the Background

Methods for inhibiting expression of a target gene in a cell, tissue, or individual organism include a method (hereinafter, sometimes referred to as “RNAi method”) wherein a double-stranded RNA is introduced to the cell, tissue, or individual organism to thereby accelerate degradation of mRNA having homology to its sequence and, as a result, to inhibit expression of a gene which is a template of the mRNA (hereinafter, this effect is sometimes referred to as “RNAi effect”). This technique has hitherto been reported to be effective in individuals of plants (Waterhouse, P. M., et al., Proc. Natl. Acad. Sci. USA., 95, 13959-13964 (1998)), trypanosome (Ngo, H, et al., Proc. Natl. Acad. Sci. USA., 95, 14687-14692 (1998)), hydra (Lohmann J. U., et al., Dev. Biol., 214, 211-214 (1999)), planarian (Sanchez Alvarado, et al., Proc. Natl. Acad. Sci. USA., 96, 5049-5054 (1999)), nematode (Fire, A., et al., Nature, 391, 806-811 (1998), Drosophila (Kannerdell, J. R., et al., Cell, 95, 1017-1026 (1998); Misquitta, L., et al., Proc. Natl. Acad. Sci. USA., 96, 1451-1456 (1999)).

Moreover, although the effect has been reported to be limited in vertebrates, it has been reported that use of a double-stranded RNA each having 19 nucleotides adjoined by 2-nucleotide 3′ overhangs enables exhibition of the RNAi effect in cultured cells of vertebrates (Elbashir, S., et al., Nature, 411, 494-498 (2001)). In identification of the gene function described below and the screening method of cell lines suitable for useful substance production, superiority of the use of the RNAi method is apparent but there are problems in that RNA is extremely easily degraded by an ribonuclease, especially in a single-stranded state, and in that cost of production is expensive. Therefore, it has been desired to develop a highly stable polynucleotide which can be used in the RNAi method.

### SUMMARY OF THE INVENTION

An object of the present invention is to provide a method for inhibiting expression of a target gene having a substantially identical nucleotide sequence with a partial nucleotide sequence of the polynucleotide, by introducing into a cell, tissue, or individual organism with a double-stranded polynucleotide having an enhanced stability owing to incorporation of DNA.

As a result of extensive studies for achieving the above objectives, the present inventors have found that, in a Chinese hamster culture cell, CHO-KI, introduction of a double-stranded polynucleotide of a hybrid of DNA and RNA having a part of the nucleotide sequence of a luciferase gene and that with double-stranded polynucleotide of a chimera of DNA and RNA inhibit expression of the luciferase gene in the cell. Thus, the present inventors have accomplished the present invention.

Namely, according to the present invention, inventions described in the following (1) to (24) are provided.

(1) A method for inhibiting expression of a target gene, which comprises introducing into a cell, tissue, or individual organism with a double-stranded polynucleotide comprising DNA and RNA having a substantially identical nucleotide sequence with at least a partial nucleotide sequence of the target gene.

(2) The method according to (1) above, wherein the double-stranded polynucleotide comprises a self complementary single strand.

(3) The method according to (1) or (2) above, wherein the double-stranded polynucleotide is a hybrid of a DNA strand and an RNA strand.

(4) The method according to (3) above, wherein the hybrid of a DNA strand and an RNA strand comprises a sense strand DNA and an antisense strand RNA.

(5) The method according to (1) or (2) above, wherein the double-stranded polynucleotide is a chimera of DNA and RNA.

(6) The method according to any one of (1) to (5) above, wherein, in the double-stranded polynucleotide, at least an upstream partial region of the polynucleotide is RNA. (7) The method according to (6) above, wherein the upstream partial region consists of 9 to 13 nucleotides.

(8) The method according to any one of (1) to (6) above, wherein the double-stranded polynucleotide consists of 15 to 30 nucleotides and at least an upstream half region of the polynucleotide is RNA.

(9) The method according to any one of (1) to (8) above, wherein the target gene is plural.

(10) A method of analyzing the function of a gene, which comprises analyzing a phenotypic change appearing in the cell, tissue, or individual organism as a result of inhibition of expression of a target gene by the method according to any one of (1) to (9) above.

(11) A method of imparting a specific property to a cell, tissue, or individual organism which comprises inhibiting expression of a target gene using the method according to any one of (1) to (9) above.

(12) A cell, tissue, or individual organism obtainable by the method according to (11) above.

(13) A method of screening an agent for preventing and/or treating a disease associated with a target gene, which comprises adding a test substance to the cell, tissue, or individual organism according to (12) above and analyzing a property change imparted to the cell, tissue, or individual organism.

(14) A method for treating a disease associated with a target gene, comprising administering a substance obtainable by the method according to (13) above to a patient in need of such treatment.

(15) A method of producing a preventing and/or therapeutic agent for a disease associated with a target gene, which comprises pharmaceutically formulating a substance selected by the method according to (13) above.

(16) A method according to any one of (1) to (9) above, which further comprises introducing into the cell, tissue, or individual organism with an expression vector containing DNA encoding an indicator protein, and subsequently selecting and analyzing the cell, tissue, or individual organism having a quantity of a signal generated from the indicator protein of a specific strength or more.

(17) A method according to any one of (1) to (9) above, which further comprises introducing into the cell, tissue, or individual organism with an expression vector comprising DNA encoding an indicator protein and a double-stranded RNA comprising a substantially identical nucleotide sequence with at least a partial nucleotide sequence of the DNA and containing DNA into a cell, and subsequently selecting and analyzing the cell, tissue, or individual organism having a reduced quantity of a signal generated from the indicator protein.

(18) The method according to (16) or (17) above, wherein the indicator protein is a protein in which the quantity of the protein and the quantity of a signal generated from the protein change in proportion.

(19) The method according any one of (16) to (18) above, wherein the indicator protein is luciferase.

(20) A method of identifying a functional domain of RNA in an RNAi method, which comprises (i) preparing a double-stranded polynucleotide having a substantially identical nucleotide sequence with at least a partial nucleotide sequence of a target gene and comprises a chimera of DNA and RNA, (ii) introducing into a cell, tissue, or individual organism with the double-stranded polynucleotide, (iii) measuring an inhibition degree of expression of the target gene in the cell, tissue, or individual organism, and (iv) identifying a sequence which is required to be RNA for inhibiting expression of the target gene.

(21) The method according to (20) above, wherein either of the double strands of the double-stranded polynucleotide is an RNA strand.

(22) A double-stranded polynucleotide for use in the method according to any one of (1) to (11), (13), and (15) to (21) above.

(23) A method for treating a disease associated with a target gene, comprising administering a substance comprising at least the double-stranded polynucleotide according to (22) above to a patient in need of such treatment.

(24) A kit for conducting the method according to (1) to (11), (13), and (15) to (21) above, comprising at least the double-stranded polynucleotide according to (22) above.

### BRIEF DESCRIPTION OF THE DRAWINGS

FIG. 1 is a drawing illustrating sequences of the sense single strand polynucleotides consisting of 21 nucleotides and the antisense single strand polynucleotides consisting of 21 nucleotides which were used for preparation of double-stranded polynucleotides. In each sequence, the left means 5′ terminal and the right means 3′ terminal, and it is shown that a bold character part is RNA and an underlined part is DNA. FLs1 corresponds to SEQ ID NO: 2, DFLs1 corresponds to SEQ ID NO: 1, FLs1-1 corresponds to SEQ ID NO: 13, FLs1-2 corresponds to SEQ ID NO: 14, FLa2 corresponds to SEQ ID NO: 4, DFLa2 corresponds to SEQ ID NO: 15, DFLa2-1 corresponds to SEQ ID NO: 16, DFLa2-2 corresponds to SEQ ID NO: 17, DFLa2-3 corresponds to SEQ ID NO: 18,

DFLa2-4 corresponds to SEQ ID NO: 19, DFLa2-5 corresponds to SEQ ID NO: 20, DFLa2-6 corresponds to SEQ ID NO: 21, DFLa2-7 corresponds to SEQ ID NO: 22, DFLa2-8 corresponds to SEQ ID NO: 23, DFLa2-9 corresponds to SEQ ID NO: 24, and DFLa2-10 corresponds to SEQ ID NO: 25.

FIG. 2 is a drawing illustrating inhibition of expression of a luc gene in the case that CHO-KI cells are introduced with double-stranded polynucleotides of DNA-RNA hybrid.

FIG. 3 is a drawing illustrating inhibition of expression of a luc gene in the case that S2 cells are trasfected with double-stranded polynucleotides wherein each sense strand is RNA and each antisense strand is DNA-RNA chimera.

FIG. 4 is a drawing illustrating inhibition of expression of a luc gene in the case that HeLa cells and HEK293 cells are introduced with double-stranded polynucleotides wherein each antisense strand is RNA and each sense strand is DNA-RNA chimera.

FIG. 5 is a drawing illustrating inhibition of expression of a luc gene in the case that CHO-KI cells are introduced with double-stranded polynucleotides which are DNA-RNA chimera.

### BEST MODE FOR CARRYING OUT THE INVENTION

All publications, patent applications, patents, and other references mentioned herein are incorporated by reference in their entirety. The materials, methods, and examples are illustrative only and are not intended to be limiting, unless otherwise specified.

The following will describe the present invention in more detail.

### (1) A Double-Stranded Polynucleotide Comprising DNA and RNA for the RNAi Method.

The present invention is a method for inhibiting expression of a target gene, which comprises introducing into a cell, tissue, or individual organism with a double-stranded polynucleotide comprising DNA and RNA having a substantially identical nucleotide sequence with at least a partial nucleotide sequence of a target gene.

In the present invention, the target gene may be any gene as long as it can produce mRNA and can optionally be translated into a protein in the cell, tissue, or individual organism to be introduced (hereinafter, this is sometimes referred to as “recipient”). Specifically, it may be endogenous to the object to be introduced or a transgene. Moreover, it may be a gene located on a chromosome or an extrachromosomal one. Examples of the extrachromosomal one include those derived from pathogens, such as viruses, bacteria, fungi, or protozoa. The function may be known or unknown or the function may be known in a cell of other organism but is unknown in the recipient.

The double-stranded polynucleotide comprising DNA and RNA having a substantially identical nucleotide sequence with at least a partial nucleotide sequence of these genes (hereinafter, this is sometimes referred to as “double-stranded polynucleotide”) comprises a substantially identical nucleotide sequence with a sequence having 20 nucleotides or more which may be any part of the nucleotide sequence of the target gene. Herein, “substantially identical” means that the sequence has homology of 50% or more, preferably 70% or more, more preferably 80% or more to the sequence of the target gene. The strand length of the nucleotide may be any length of 19 nucleotides to the full length of the open reading frame (ORF) of the target gene, but those having a strand length of 19 to 500 nucleotides are preferably used. However, in cells derived from mammals, there is known the presence of a signal transduction system which is activated in response to a double-stranded RNA having a strand length of 30 nucleotides or more. This is called a interferon response (Mareus, P. I. et at., Interferon, 5, 115-180 (1983)). When such a double-stranded RNA is introduced into a cell, start of translation of many genes is non-specifically inhibited through PKR (dsRNA-responsive protein kinese: Bass, B. L., Nature, 411, 428-429 (2001)) and at the same time, RNaseL is activated through 2′,5′ oligoadenylate synthetase (Bass, B. L., Nature, 411, 428-429 (2001)) to cause non-specific degradation of RNA in the cell. Owing to these non-specific reactions, a specific reaction on the target gene is concealed. Therefore, in the case of using a mammal or a cell or tissue derived from said mammal as a recipient, a double-stranded polynucleotide consisting of 15 to 30, preferably 19 to 25, more preferably 19 to 23, and most preferably 19 to 21 nucleotides is used. The double-stranded polynucleotide of the present invention is not necessarily double-stranded over the whole length and includes parts having 5′- or 3′-overhang, and the overhang terminal may be from 1 to 5 nucleotides, preferably from 1 to 3 nucleotides, and more preferably 2 nucleotides. Moreover, the most preferable example includes one having a structure containing 3′-overhang of 2 nucleotides at 3′-terminal of each polynucleotide strand. A double-stranded polynucleotide means a polynucleotide in which a part having complementarity is double-stranded but may be a polynucleotide in which a self-complementary single strand polynucleotide is self-annealed. Examples of the self-complementary single strand polynucleotide include those having, e.g., an inverted repeat.

Furthermore, when mixing of DNA and RNA, a hybrid type of a DNA strand and an RNA strand or a chimera type of DNA and RNA is used. The hybrid of a DNA strand and an RNA strand may be any one as far as it has an activity to inhibit expression of the target gene when a recipient is introduced with it, but preferably, one in which the sense strand is DNA and the antisense strand is RNA is used. Also, the chimera type of DNA and RNA may be any one as far as it has an activity to inhibit expression of the target gene when a recipient is introduced with it. In order to enhance stability of the double-stranded polynucleotide, it is preferable to contain DNA as much as possible. However, among the chimera type double-stranded polynucleotides of the present invention, it is preferable to suitably determine a sequence which is required to be RNA for inhibiting expression of the target gene within the range where the inhibition of the expression occurs with carrying out analysis of an inhibition degree of expression of the target gene as described below. Thereby, a functional domain of RNA in the RNAi method can be also identified. As a preferred example of the chimera type thus determined, one in which an upstream partial region of the double-stranded polynucleotide is RNA may be mentioned, for example. Herein, the upstream partial region means 5′ side of the sense strand and 3′ side of the antisense strand. The upstream partial region preferably means a domain of 9 to 13 nucleotides from the terminal of the upstream of the above double-stranded polynucleotide. Moreover, preferred examples of a such chimera type double-stranded polynucleotide include a double-stranded polynucleotide having a strand length of 19 to 21 nucleotides in which at least an upstream half region of the polynucleotide is RNA and the rest is DNA.

The method of preparing the double-stranded polynucleotide is not particularly limited but it is preferable to use a known chemical synthetic method. In the chemical synthesis, complementary single-stranded polynucleotides are separately synthesized and are annealed with each other by an appropriate method, whereby a double-stranded form can be obtained. A specific example of the method for the annealing include a method wherein the synthesized single-stranded polynucleotides are mixed in a molar ratio of preferably at least about 3:7, more preferably about 4:6, and most preferably substantially equimolar amount (e.g., a molar ratio of about 5:5) and heated to a temperature at which the double-stranded oform is dissociated and then the mixture is gradually cooled. The annealed double-stranded polynucleotide is purified by a known usually employed method, if necessary. An example of usable purification methods include a method wherein confirmation with an agarose gel is conducted and the remaining single-stranded polynucleotide is optionally removed by, for example, degradation with an appropriate enzyme.

Moreover, in the case that a single-stranded polynucleotide having an inverted repeat is prepared as the single-stranded polynucleotide having self complementarity, the polynucleotide is prepared by a method of chemical synthesis and then a self complementary sequence is annealed in the same manner as described above.

(2) Introduction of a Cell, Tissue, or Individual Organism with a Double-Stranded Polynucleotide, and Inhibition of Expression of a Target Gene.

A recipient for the introduction of the double-stranded polynucleotide thus prepared may be any recipient as far as the target gene can be transcribed into RNA or translated into a protein in the cell. Specifically, the recipient in the present invention means a cell, tissue, or individual organism. The cell in the present invention may be any cell from the germ line or somatic cell, totipotent or pluripotent cell, dividing or non-dividing cell, parenchyma or epithelium cell, or immortalized or transformed cell. Specifically, the cell may be an undifferentiated cell such as a stem cell, cell derived from organs or tissues, or differentiated cells thereof. The tissue includes single cell embryos or constitutional cells, or multi cell embryos, and fetal tissues. Moreover, examples of the above differentiated cells include adipocytes, fibroblasts, myocytes, cardiomyocytes, endothelium, neurons, glia, blood cells, megakaryocytes, lymphocytes, macrophages, neutrophils, eosinophils, basophils, mast cells, leukocytes, granulocytes, keratinocytes, chondrocytes, osteoblasts, osteoclasts, hepatocytes, and cells of the endocrine or exocrine glands. As a specific example of such cells, a CHO-KI cell (RIKEN Cell bank), a Drosophila S2 cell (Schneider, I., et al., J. Embryol. Exp. Morph., 27, 353-365 (1972)), a human HeLa cell (ATCC:CCL-2), a human HEK293 cell (ATCC: CRL-1573), are preferably used.

Furthermore, an individual organism to be used as a recipient in the present invention specifically includes organisms belonging to a plant, animal, protozoan, bacterium, virus, or fungus. The plant may be a monocot, dicot or gymnosperm; the animal may be a vertebrate or invertebrate. Preferred microbes as the recipients of the present invention are those used in agriculture or by industry, and those that are pathogenic for plants or animals. Fungi include organisms in both the mold and yeast morphologies. Examples of vertebrate animals include fishe and mammals, such as cattle, goat, pig, sheep, hamster, mouse, rat, monkey, and human; invertebrate animals include nematodes and other worms, Drosophila, and other insects.

As a method of introducing the double-stranded polynucleotide into a recipient, a calcium phosphate method, an electroporation method, a lipofection method, a viral infection, an immersion in a double-stranded polynucleotide solution, or a transformation method is used, when the recipient is a cell or a tissue. Moreover, as a method of introduction into an embryo, microinjection, an elecroporation method, or a viral infection may be mentioned. When the recipient is a plant, a method of injection or perfusion into a cavity or an interstitial cell of the plant or spraying may be used. In the case of an animal individual, a method of systemic introduction, such as oral, topical, parenteral (inclusive of subcutaneous, intramuscular, and intravenous administration), transvaginal, transrectal, intranasal, transocular, or intraperitoneal administration, or an electroporation method or a viral infection may be used. As the method for oral introduction, a double-stranded polynucleotide can be directly mixed with food for the organism. Furthermore, in the case of introduction into an individual organism, introduction can be also conducted by administration as an embedded long-term releasing preparation or by ingestion of an introduced recipient into which the double-stranded polynucleotide has been introduced, for example.

The quantity of the double-stranded polynucleotide for introduction may be suitably selected depending on the recipient and the target gene, but it is preferable to introduce the polynucleotide in an amount sufficient to introduce at least one copy per cell. Specifically, when the recipient is a human cultured cell and the double-stranded polynucleotide is introduced by a calcium phosphate method, for example, the amount is preferably 0.1 to 1000 nM.

Two or more kinds of the double-stranded polynucleotides may be also introduced simultaneously. In this case, inhibition of expression of two or more target genes is expected in the cell, tissue, or individual organism introduced with the polynucleotides (hereinafter, this is referred to as “introduced recipient”).

In the present invention, “inhibition of expression of the target gene” means not only complete inhibition of the expression but also inhibition of 20% or more as an expressed amount of mRNA or a protein. The inhibition degree of expression of the target gene can be measured by comparing the accumulation of RNA of the target gene or produced amounts of the protein encoded by the target gene in the double-stranded polynucleotide-introduced recipient and non-introduced recipient. The amount of mRNA can be measured by a known usually employed method. For example, it can be conducted by the Northern hybridization analysis, the quantitative reverse transcription PCR, or in situ hybridization. Moreover, the produced amount of the protein can be measured by a Western blot analysis or by determining an enzymatic activity of the protein encoded by the target gene.

### (3) A Method of Analyzing a Gene Function by Inhibiting Expression of a Gene in the Introduced Recipient.

By analyzing a phenotypic change appearing in the introduced recipient as a result of the inhibition of expression of a gene in the introduced recipient by the double-stranded polynucleotide according to the present invention, it is possible to identify a function of the gene targeted by the introduced double-stranded polynucleotide.

Herein, the target gene may be a gene which function is known or a gene which function in the recipient is unknown. The double-stranded polynucleotide corresponding to the target gene is prepared as described in (1) above and is introduced into the recipient described in (2) in a similar manner. The changed phenotype to be analyzed in the introduced recipient is not particularly limited and examples thereof include performances of an organism, such as morphology of the introduced recipient, an amount of a substance in the introduced recipient, an amount of a substance secreted by the introduced recipient, dynamic behavior of a substance in the introduced recipient, adhesion between the introduced recipients, mobility of the introduced recipient, or life of the introduced recipient. When the function of the target gene is known in the other recipient, it is preferable to analyze a phenotype related to the function. As a means for analyzing the phenotypic change, in the case of analyzing morphological change of the introduced recipient, it is possible to use a method of a microscopic or visual detection. Moreover, when mRNA as a substance in the introduced recipient, methods of analyzing its amount include, for example, Northern hybridization, quantitative reverse transcription PCR, or in situ hybridization. In the case of a protein, methods of analyzing the amount include a Western blot analysis with an antibody using a protein encoded by the target gene as an antigen, a method of measuring an enzymatic activity of the protein encoded by the target gene. Since the phenotypic change appearing only in the introduced recipient thus analyzed occurs as a result of the inhibition of expression of the target gene, it can be identified as a function of the target gene.

### (4) A Method of Imparting a Specific Property to a Cell, Tissue, or Individual Organism by Inhibiting Expression of a Target Gene Using a Double-Stranded Polynucleotide.

By inhibiting expression of the target gene using the double-stranded polynucleotide of the present invention, a specific property can be imparted to a cell, tissue, or individual organism. The specific property means a property appearing in the introduced recipient as a result of the inhibition of expression of the target gene. The target gene herein may be a gene wherein a property imparted to the introduced recipient by the inhibition of its expression is already clarified or the function thereof or the function in the introduced recipient is unknown. With regard to the target gene which function is unknown, by selecting a desired phenotype among the phenotypes exhibited by the introduced recipient after the introduction of the double-stranded polynucleotide thereto, a desired property can be imparted to the introduced recipient.

Specific examples of the desired property to be imparted to the introduced recipient include an intracellular productive function, a function of inhibiting extracellular secretion, a repairing function of an injury of a cell or DNA, and a resistant function to a specific disease. Specifically, when the introduced recipient is a plant individual, the target gene includes genes associated with enzymes relating to fruit ripening, plant structural proteins, or pathogenicity.

The case that the inhibition of expression of the target gene has a resistant function to a specific disease is a case that increase of expression of a specific protein becomes a cause of the specific disease and the target gene includes a gene encoding the above protein and a gene encoding a protein having a function of controlling expression of the above protein. As a specific example, the target gene is a gene necessary for retention of a carcinogenic/tumorigenic phenotype and the recipient is a cancerous cell or a tumor tissue.

Since the double-stranded polynucleotide toward such a target gene inhibits expression of the protein encoded by the target gene, the polynucleotide can be used as an agent for treating or preventing diseases associated with the target gene. When the double-stranded polynucleotide is used as an active ingredient of the above pharmaceutical agent, the polynucleotide may be used solely but may also be used as a pharmaceutical composition formulated with a pharmaceutically acceptable carrier. The ratio of the active ingredient relative to the carrier at this time may vary between 1 to 90% by weight. Moreover, such a pharmaceutical agent can be administered in various forms which include oral administration with tablets, capsules, granules, powders, or syrups, or parenteral administration with injections, drips, liposomes, or suppositories. In addition, its dose can be suitably selected depending on symptoms, age, body weight, and other factors.

The introduced recipient with the double-stranded polynucleotide targeting such a gene is selected depending on a phenotype predicted to be associated with the inhibition of expression of the gene. Moreover, in the double-stranded polynucleotide for introduction, when a sequence encoding a specific genetic marker, e.g., a fluorescent protein, is connected, the selection is possible based on an inhibition degree of expression of the fluorescent protein introduced together with the double-stranded polynucleotide into the recipient. When a gene functioning for cancer suppression is used as a target gene, cell characters to be selected include characters of malignant tumor, such as increase of the growth ability, decrease of the cell adhesion ability, or increase of the motile (metastatic) ability. Moreover, when a gene controlling biological rhythm is used as a target gene, cell characters to be selected include, e.g., disappearance of circadian rhythm. Furthermore, when a gene involved in repair of a DNA injury induced by an environmental mutagen is used as a target gene, cell characters to be selected include, e.g., exhibition of sensitivity toward the mutagen.

The selected introduced recipient can be established and obtained as a line by a known cloning technology, which is suitable to each recipient. For example, when the recipient is a cell, the introduced recipient can be established and obtained as a cell line by a limiting dilution method or a method with a drug-resistant marker, which are cell line-establishing methods in usual cultured cells. The introduced recipient, which is obtainable in the present invention and to which a specific function is imparted, can be used as a cell line having an increased efficiency of producing or secreting a useful substance, a cell line exhibiting a high sensitivity to an environmental factor providing an injury against cells or DNA, or a model for treating a disease, which exhibits a character associated with the disease.

The method of obtaining a cell line to be a model for treating a disease will be described as a further specific applied example of the present invention. As the target gene, a gene which decrease in the expressed amount or which deficiency becomes a cause of a disease may be mentioned. Specifically, there may be mentioned PS1 gene in Alzheimer's disease, XPA/XPD/XPF/XPG genes and DNA polymerase η gene in xeroderma pigmentosum syndrome, APC gene in colon cancer, BRCA1/BRCA2 genes in breast cancer, INS/INSR genes in diabetes, and other examples.

By introducing, for example, a human-derived cultured cell with a double-stranded polynucleotide comprising DNA and RNA having a substantially identical nucleotide sequence with at least a partial nucleotide sequence of these human genes, a human disease model cell can be obtained.

Furthermore, by bringing a test substance into contact with the cell, tissue, or individual organism to which the specific property is imparted and by analyzing whether symptoms of the disease associated with the gene or a change in character appears or not, it is also possible to screen an agent for treating and/or preventing the above disease.

When the substance selected by such a screening is used as an active ingredient of the above pharmaceutical agent, the substance can be used solely but can be also used as a pharmaceutical composition formulated with a pharmaceutically acceptable carrier. The ratio of the active ingredient relative to the carrier at this time may vary between 1 to 90% by weight. Moreover, such a pharmaceutical agent can be administered in various forms and the administration forms include oral administration with tablets, capsules, granules, powders, or syrups, or parenteral administration with injections, drips, liposomes, or suppositories. In addition, its dose can be suitably selected depending on symptoms, age, body weight, and the other factors.

### (5) A Method of Use of the Primary Selection Using an Inhibition Degree of Expression of an Indicator Gene as an Index

The methods of the present invention described in the above (1) to (4) are methods of introducing a recipient with a double-stranded polynucleotide comprising DNA and RNA having a substantially identical nucleotide sequence with at least a partial nucleotide sequence of a target gene. However, in the method of the present invention, by further introducing (a) an expression vector comprising DNA encoding an indicator protein, (b) a double-stranded polynucleotide comprising DNA and RNA having a substantially identical nucleotide sequence with at least a partial nucleotide sequence encoding the indicator protein, and primarily screening the introduced recipient using a quantity of a signal generated from the indicator protein as a measure, only a introduced recipient in which expression of the gene in the introduced recipient is inhibited can be analyzed, so that an efficient analysis can be conducted.

As a further specific example of the present invention, the following will describe a case that a cultured cell derived from a vertebrate animal is used as a recipient and a fluorescent protein is used as an indicator protein. A cultured cell derived from a vertebrate animal is introduced with an expression vector comprising DNA encoding a fluorescent protein, and the cell is cultured, followed by selection of a cell having a quantity of signal generated from the indicator protein of a specific strength or more. The cell selected herein is further introduced with a double-stranded polynucleotide comprising DNA and RNA having a substantially identical nucleotide sequence with at least a partial nucleotide sequence of DNA encoding the indicator protein, and the cell is cultured, followed by analysis of an inhibition degree of expression of the indicator gene based on a degree of attenuation of the fluorescence generated from the indicator protein.

Since each primary screening confirms the introduction of the recipient with the double-stranded and occurrence of the inhibition of expression of the target gene in the introduced recipient, the indicator protein should be a protein which shows a correlation between the amount of the protein and the quantity of signal generated therefrom. Specific examples of such a protein include luciferase protein.

Furthermore, in measuring the inhibition degree of expression of the target gene, it is also possible to calculate the amount of the protein encoded by the target gene based on the expressed amount of the indicator protein.

### (6) A Kit for Use in the Present Invention.

The kit for conducting the methods described in (1) to (5) above contains a double-stranded polynucleotide, a vector comprising DNA encoding an indicator protein, a double-stranded polynucleotide comprising DNA and RNA having a substantially identical nucleotide sequence with at least a partial nucleotide sequence of the indicator gene, reagents such as enzymes and buffers, and reagents for introducing polynucleotides. It is not necessary for the kit of the present invention to contain all these reagents and the kit may contain any combination of the reagents, and possibly additional components, as far as the kit can be used in the above methods of the present invention.

### EXAMPLES

The following describes the present invention more specifically with reference to Examples but the following Examples should be construed as a help for obtaining a specific recognition of the present invention and hence the scope of the present invention is by no means limited by the following Examples.

### Example 1

### Inhibition Of Expression of a Target Gene by a Double-Stranded DNA-RNA Hybrid Introduced into CHO-KI Cells.

### (1) Preparation of the DNA-RNA Hybrid Type Double-Stranded Polynucleotide.

A luciferase gene of Photinus pyralis (P. pyralis luc gene: accession number: U47296) was used as a target gene and pGL3-Control vector (manufactured by Promega) was used as an expression vector comprising the same. The gene fragment of P. pyralis luc gene lies between a promoter of SV40 and poly A signal in the vector. A luciferase gene of Renilla reniformis was used as an indicator gene and pRL-TK (manufactured by Promega) was used as an expression vector comprising the same.

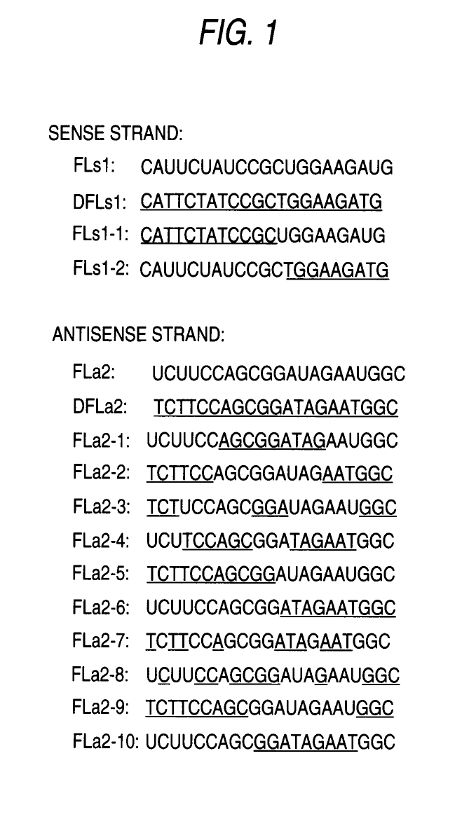

The sense strand of 21 nucleotides for use in the preparation of the double-stranded polynucleotide used in the present Example is represented by SEQ ID NO: 1 (DNA) or SEQ ID NO: 2 (RNA). Moreover, the antisense strand is represented by SEQ ID NO: 3 (DNA) or SEQ ID NO: 4 (RNA). With regard to these sequences, a chimera type single-stranded polynucleotide of DNA or RNA was prepared as shown in FIG. 1. Synthesis of these polynucleotides was entrusted to Genset K. K. via Hitachi Instruments Service Co., Ltd. The sequence of the sense strand polynucleotide corresponds to 38th to 58th nucleotides of the P. pyralis luc gene (total length of 1,653 base pairs), which is a target gene in pGL3-Control vector.

The double-stranded RNA, double-stranded DNA and double-stranded DNA-RNA hybrid used for inhibiting expression of the P. pyralis luc gene were prepared by annealing the sense strand FLs1 (RNA) or DFLs1 (DNA) with the antisense strand Fla2 (RNA) or DFLa2 (DNA). The annealing was conducted by heating the sense single-stranded polynucleotide and the antisense single-stranded polynucleotide in a 10 mM Tris-HCl (pH 7.5) and 20 mM NaCl reaction liquid at 90° C. for 2 minutes and further incubating the whole at 37° C. for 1 hour, followed by allowing it to stand until it was cooled to room temperature. Thereby, the sense strand and the antisense strand were annealed to form a double-stranded polynucleotide. The formation of the double-stranded polynucleotide was assayed by an electrophoresis on 2% agarose gel in TBE buffer. Under the above conditions, almost every single-stranded polynucleotide was annealed into a double-stranded polynucleotide.

(2) Introduction of a Target Gene, Indicator Gene, and Double-Stranded Polynucleotide into Cultured Cells.

CHO-KI cells (RIKEN Cell Bank) were used as a cultured cell and Dulbecco's modified Eagle's medium (manufactured by Gibco BRL) supplemented with inactivated 10% bovine fetal serum (manufactured by Mitsubishi Kasei Corporation) and 10 units/ml penicillin (manufactured by Meiji) and 50 μg/ml streptomycin (manufactured by Meiji) as antibiotics was used as a medium. The cells were cultured at 37° C. in the presence of 5% CO₂.

The CHO-KI cells were spread on a 24-well plate at a concentration of 0.3×10⁶ cells/ml. After 1 day, 1.0 μg pGL3-Control DNA, 0.5 μg pRL-TK DNA, and 0.01, 0.1, 1, 10, or 100 nM of each double-stranded polynucleotide were introduced by Ca-phosphate precipitation method (Saibo Kogaku Handbook, edited by Toshio Kuroki et al., Youdosha (1992)).

### (3) Measurement of Gene Expression in Cultured Cells.

The cells prepared in Example 1 (2) above were collected after 20 hours and expressed amounts (luciferase activity) of two kinds of luciferase (Photinus pyralis luc and Renilla reniformis luc) proteins were measured using Dual-Luciferase Reporter Assay System (manufactured by Promega). The measurement of fluorescence was conducted using a Lumat LB9507 luminometer (EG&G Berthold).

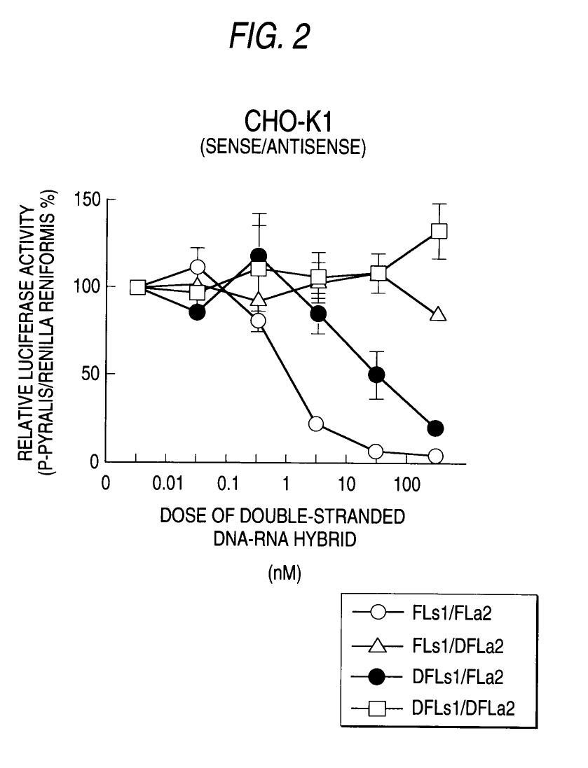

The expression of the gene introduced into CHO-KI cells was inhibited by the DNA-RNA hybrid type double-stranded polynucleotides (FIG. 2). All of the values show a relative activity of target gene products toward expressed amounts of the indicator gene (enzymatic activity of luciferase). FIG. 2 shows the average values of three-time experiments and vertical bars in the figure represent standard deviations. As compared with control groups in which no double-stranded polynucleotide was introduced, 96% inhibition of the expression of the target gene was observed in the double-stranded RNA introduced groups when the double-stranded polynucleotide was added in an amount of 100 nM and 80% inhibition was observed in the group in which the double-stranded polynucleotide having a sense strand DNA and an antisense strand RNA was introduced. Even when the sense side of the double-stranded polynucleotide was DNA, it was proved that the gene expression can be inhibited in CHO-KI cells when the antisense side was RNA, although the effect was weak as compared with the double-stranded RNA.

### Example 2

### Inhibition of Expression of a Target Gene by a DNA-RNA Chimera Type Double-Stranded Polynucleotide Introduced into Drosophila S2 Cells

### (1) Preparation of the DNA-RNA Chimera Type Double-Stranded Polynucleotide.

A luciferase gene of Photinus pyralis (P. pyralis luc gene: accession number: U47296) was used as a target gene. Moreover, a luciferase gene of Renilla reniformis was used as an indicator gene. Furthermore, the expression vectors described in Example 1 were also used as expression vectors.

The sense strand of 21 nucleotides for use in the preparation of the double-stranded polynucleotide used in the present Example is represented by SEQ ID NO: 1 (DNA) or SEQ ID NO: 2 (RNA). Moreover, the antisense strand is represented by SEQ ID NO: 3 (DNA) or SEQ ID NO: 4 (RNA). With regard to these sequences, chimera type single-stranded polynucleotides of DNA or RNA were prepared as shown in FIG. 1. The synthesis of these polynucleotides was entrusted to Genset K. K. via Hitachi Instruments Service Co., Ltd.

The DNA-RNA chimera type double-stranded polynucleotides used for inhibiting expression of the P. pyralis luc gene were prepared by annealing the sense strand FLs1 (single-stranded RNA) with the antisense strands Fla2-1, Fla2-2, Fla2-3, Fla2-4, Fla2-5, Fla2-6, Fla2-7, Fla2-8, Fla2-9, and Fla2-10 (DNA-RNA chimera type single-stranded polynucleotides), respectively. The annealing was conducted by reacting the sense single-stranded RNA and the antisense DNA-RNA-chimera type single-stranded polynucleotide in a manner similar to Example 1. The production of the double-stranded polynucleotide was assayed by an electrophoresis on 2% agarose gel in TBE buffer.

(2) Introduction of a Target Gene, Indicator Gene, and a DNA-RNA Chimera Type Double-Stranded Polynucleotide into Cultured Cells.

Drosophila S2 cells (Schneider, I., et al., J. Embryol. Exp. Morph., 27, 353-365 (1972)) were used as a cultured cell and Schneider's modified Eagle's medium (manufactured by Gibco BRL) supplemented with inactivated 10% bovine fetal serum (manufactured by Mitsubishi Kasei Corporation) and 10 units/ml penicillin (manufactured by Meiji) and 50 μg/ml streptomycin (manufactured by Meiji) as antibiotics was used as a medium. The cells were cultured at 25° C. in the presence of 5% CO₂.

The S2 cells were spread on a 24-well plate at a concentration of 1.0×10⁶ cells/ml. After 1 day, 1.0 μg pGL3-Control DNA, 0.05 μg pRL-TK DNA, and 100 nM of each double-stranded polynucleotide were introduced by Ca-phosphate precipitation method (Saibo Kogaku Handbook, edited by Toshio Kuroki et al., Youdosha (1992)).

(3) Measurement of gene expression in cultured cells.

The cells prepared in the Example 2 (2) above were collected after 20 hours and expressed amounts of two kinds of luciferase proteins were measured using Dual-Luciferase Reporter Assay System (manufactured by Promega). The measurement of fluorescence was conducted using a Lumat LB9507 luminometer (EG&G Berthold).

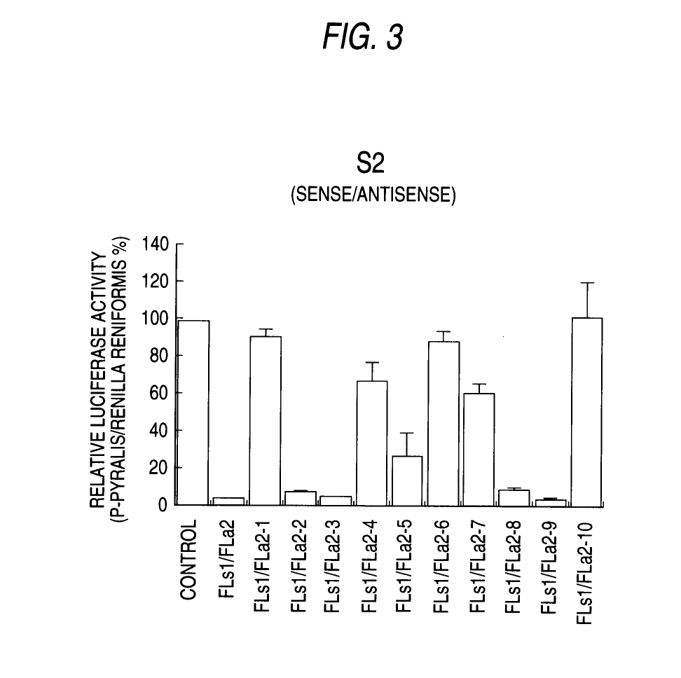

The expression of the gene introduced into S2 cells was inhibited by the DNA-RNA chimera type double-stranded polynucleotides of 21 nucleotides and prepared so that the sense side was fixed to RNA and the antisense side was a chimera type polynucleotide of DNA and RNA (FIG. 3). All the values were determined as relative activity of target gene products toward expressed amounts of the indicator genes (luciferase activity) and the values were shown as average values of three-time experiments and standard deviations. As compared with the control groups in which no double-stranded polynucleotide was introduced, the double-stranded polynucleotide with the antisense side Fla2, Fla2-2, Fla2-3, Fla2-8, or Fla2-9 strongly inhibited the gene expression to an approximately equal degree of 96%, 92%, 94%, 91%, or 96%, respectively. The polynucleotide having the antisense side of Fla2-5 exhibited a relatively strong inhibitory effect of 73%. The polynucleotides having the antisense side of Fla2-1, Fla2-4, Fla2-6, Fla2-7, or Fla2-10 exhibited no inhibitory effect or extremely weak effect even when the inhibition was observed. One characteristic common to the double-stranded polynucleotides exhibiting a strong effect is that two nucleotides at 13th and 14th positions from 5′ terminal on the sequence of the antisense strand are RNA (UA). On the other hand, in the double-stranded polynucleotide exhibiting a weak effect, both of the corresponding two nucleotides on the sequence of the antisense strand were DNA (TA). Therefore, in the present Example using S2 cells, it is suggested that these two nucleotides are a domain necessary and sufficient for exhibiting a strong RNAi effect.

In the present Example, the RNAi effect was observed even when introduced with a double-stranded polynucleotide in which the part containing this domain on the antisense sequence was reserved as RNA and the other part was substituted with DNA. The double-stranded polynucleotides of 21 nucleotides and prepared by identifying or predicting a domain necessary and sufficient for exhibition of the RNAi effect according to the method as used in the present Example and the other technique, reserving a part containing this domain as RNA, and substituting the other part with DNA are considered to be able to inhibit expression of the target gene by the RNAi effect.

### Example 3

### Inhibition of Gene Expression by a DNA-RNA Chimera Double-Stranded Polynucleotide Introduced into Human Hela Cell and Human HEK293 Cells

### (1) Preparation of the DNA-RNA Chimera Type Double-Stranded Polynucleotide.

P. pyralis luc gene was used as a target gene and pGL3-Control vector (manufactured by Promega) was used as an expression vector containing the same. Moreover, a luc gene of Renilla reniformis was used as an indicator gene and pRL-TK (manufactured by Promega) was used as an expression vector containing the same.

The sense strand of 21 nucleotides for use in the preparation of the double-stranded polynucleotide used in the present Example is represented by SEQ ID NO: 1 (DNA) or SEQ ID NO. 2 (RNA). Moreover, the antisense strand is represented by SEQ ID NO: 3 (DNA) or SEQ ID NO: 4 (RNA). With regard to these sequences, chimera type single-stranded polynucleotides of DNA or RNA were prepared as shown in FIG. 1. The synthesis of these polynucleotides was entrusted to Genset K.K. via Hitachi Instruments Service, Co., Ltd. The sequence of the sense strand polynucleotide corresponds to 38th to 58th nucleotides of the P. pyralis luc gene (total length of 1,653 base pairs), which is a target gene in pGL3-Control vector.

The DNA-RNA chimera type double-stranded polynucleotides used for inhibiting expression of the P. pyralis luc gene were prepared by annealing the sense strand FLs1-1 or FLs1-2 (DNA-RNA chimera type single-stranded polynucleotide) with the antisense strand Fla2 (single-stranded RNA). The annealing was conducted by reacting the sense DNA-RNA-chimera type single-stranded polynucleotide and the antisense single-stranded RNA in a manner similar to Example 1. The production of the double-stranded polynucleotide was assayed by an electrophoresis on 2% agarose gel in TBE buffer.

(2) Introduction of a Target Gene, Indicator Gene, and a DNA-RNA-Chimera Type Double-Stranded Polynucleotide into Cultured Cells.

The pGL3-Control described in (1) above was used as a recombinant expression vector for expressing the target gene and pRL-TK described in (1) above was used as an expression vector of the indicator gene. Human HeLa cells (ATCC: CCL-2) and human HEK293 cells (ATCC: CRL-1573) were used as cultured cells and Dulbecco's modified Eagle's medium (manufactured by Gibco BRL) supplemented with inactivated 10% bovine fetal serum (manufactured by Mitsubishi Kasei Corporation) and 10 units/ml penicillin (manufactured by Meiji) and 50 μg/ml streptomycin (manufactured by Meiji) as antibiotics was used as a medium. The cells were cultured at 37° C. in the presence of 5% CO₂. The HeLa cells and HEK293 cells were spread on a 24-well plate at a concentration of 0.5×10⁶ cells/ml and 0.25×10⁶ cells/ml, respectively. After 1 day, 1.0 μg pGL3-Control DNA, 1.0 pg pRL-TK DNA, and 100 nM of each DNA-RNA chimera type double-stranded polynucleotide were introduced by Ca-phosphate precipitation method.

### (3) Measurement of Gene Expression in Cultured Cells.

As in Example 1, the cells prepared in Example 3 (2) above were collected after 20 hours and expressed amounts of two kinds of luciferase proteins were measured using Dual-Luciferase Reporter Assay System. The measurement of fluorescence was conducted using a Lumat LB9507 luminometer.

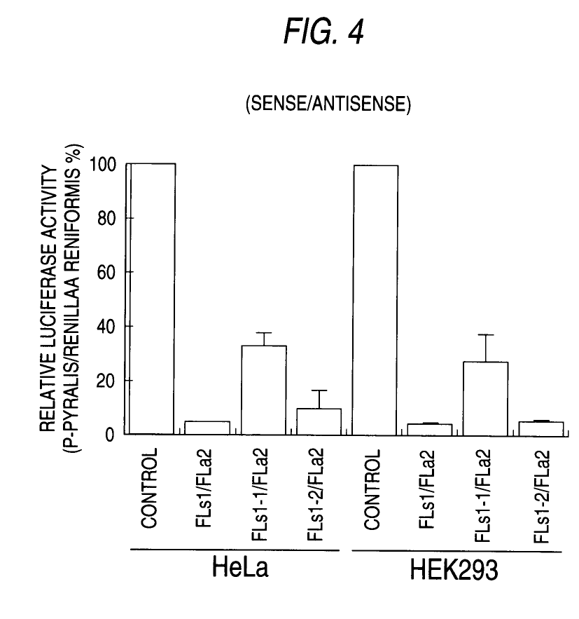

The expression of the gene introduced into HeLa cells and HEK293 cells was inhibited by the double-stranded polynucleotides of 21 nucleotides and prepared so that the antisense side of the double strand was fixed to RNA and the sense side was prepared as a chimera polynucleotide of DNA and RNA (FIG. 4). All the values were determined as relative activity of the target gene products toward expressed amounts of the indicator genes (enzymatic activity of luciferase) and the values were shown as average values of three-time experiments and standard deviations. As compared with the control groups in which no double-stranded polynucleotide was introduced, the groups in which the polynucleotides having the sense side of FLs1-2 among the double-stranded polynucleotides was introduced exhibited a strong inhibitory effect of 90% (HeLa cells) or 95% (HEK293 cells), which was equal to the case of the groups in which the double-stranded RNA was introduced. In FLs1-2, 12 nucleotides in the 5′ region of the sequence are RNA and nucleotides in the other domain are DNA. Therefore, in the present Example using HeLa cells and HEK293 cells, it is suggested that the whole of the 12 nucleotides in the sense strand or a part thereof is a domain necessary and sufficient for exhibition of a strong RNAi effect when the antisense strand is fixed to RNA.

### Example 4

### Inhibition of Gene Expression by a DNA-RNA Chimera Type Double-Stranded Polynucleotide Introduced into CHO-KI Cells

### (1) Preparation of the DNA-RNA Chimera Type Double-Stranded Polynucleotide.

P. pyralis luc gene was used as a target gene and pGL3-Control vector (manufactured by Promega) was used as an expression vector containing the same. Moreover, a luc gene of Renilla reniformis was used as an indicator gene and pRL-TK (manufactured by Promega) was used as an expression vector comprising the same.

The sequence of the sense strand of 21 nucleotides for use in the preparation of the double-stranded polynucleotide used in the present Example corresponds to polynucleotides of 8th to 28th (8-28), 38th to 58th (38-58), and 1087th to 1107th (1087-1107) of P. pyralis luc gene (total length of 1,653 base pairs), which is a target gene in the pGL3-Control vector.

With regard to each base sequence, the sense strand of 8-28 is represented by SEQ ID NO: 5 (DNA) or 6 (RNA) and antisense strand thereof is represented by SEQ ID NO: 7 (DNA) or 8 (RNA). The sense strand of 38-58 is one represented by SEQ ID NO: 1 (DNA) or 2 (RNA) and the antisense strand thereof is represented by SEQ ID NO: 3 (DNA) or 4 (RNA). In addition, the sense strand of 1087-1107 is represented by SEQ ID NO: 9 (DNA) or 10 (RNA) and the antisense strand thereof is represented by SEQ ID NO: 11 (DNA) or 12 (RNA).

With regard to these sequences, there were prepared one in which about an upstream half region(10 to 13 nucleotides) is RNA both for sense and antisense (C), one in which about an upstream half region (10 to 13 nucleotides) is DNA both for sense and antisense (D), one in which antisense is RNA and about an upstream half region (10 to 13 nucleotides) of the sense strand is RNA (E), and one in which sense strand is RNA and about an upstream half region (10 to 13 nucleotides) of the antisense strand is DNA (F). Synthesis of these polynucleotides was entrusted to Genset K. K. via Hitachi Instruments Service, Co., Ltd. and, after formation of chimera type polynucleotides, double stranded one was prepared by annealing each of them. The annealing was conducted by reacting the sense and antisense chimera single-stranded polynucleotides in a similar manner to Example 1. The production of the double-stranded polynucleotide was assayed by an electrophoresis on 2% agarose gel in TBE buffer.

(2) Introduction of a Target Gene, Indicator Gene, and a DNA-RNA Chimera Type Double-Stranded Polynucleotide into Cultured Cells.

The pGL3-Control described in (1) above was used as a recombinant expression vector for expressing the target gene and pRL-TK described in (1) above was used as an expression vector of the indicator gene. CHO-KI cells (ATCC: CCL-61) were used as a cultured cell and Dulbecco's modified Eagle's medium (manufactured by Gibco BRL) supplemented with inactivated 10% bovine fetal serum (manufactured by Mitsubishi Kasei Corporation) and 10 units/ml penicillin (manufactured by Meiji) and 50 μg/ml streptomycin (manufactured by Meiji) as antibiotics was used as a medium. The cells were cultured at 37° C. in the presence of 5% CO₂.

The CHO-KI cells were spread on a 24-well plate at a concentration of 0.3×10⁶ cells/ml. After 1 day, 1.0 μg of pRL-TK DNA and 10 nM or 100 nM of each DNA-RNA chimera type double-stranded polynucleotide were introduced by Ca-phosphate precipitation method.

### (3) Measurement of Gene Expression in Cultured Cells.

As in Example 1, the cells prepared in Example 4 (2) above were collected after 20 hours and expressed amounts of two kinds of luciferase proteins were measured using Dual-Luciferase Reporter Assay System. The measurement of fluorescence was conducted using a Lumat LB9507 luminometer.

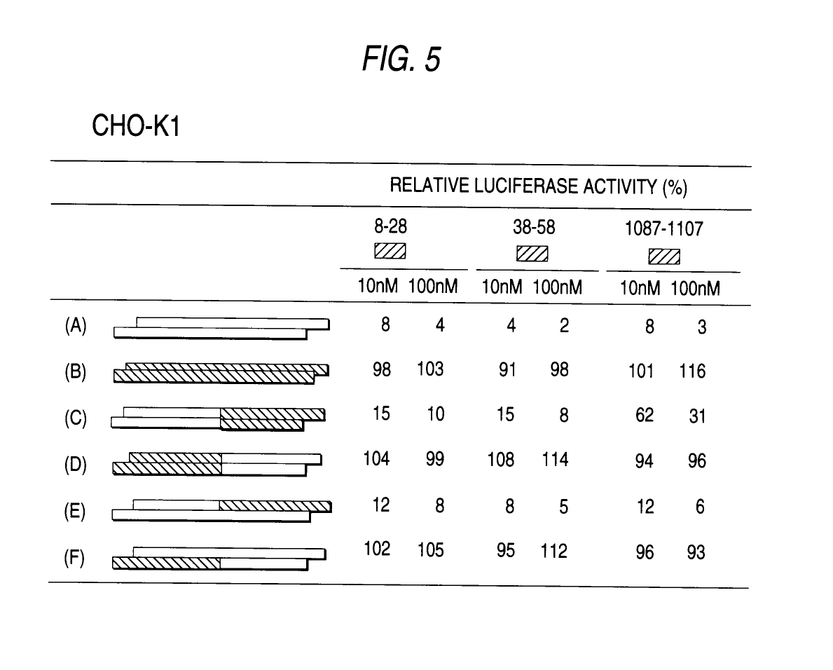

The structures of the double-stranded polynucleotides and the proportion of the luciferase activity of the cells introduced with the polynucleotides to that of a control (a cell in which no double-stranded polynucleotide was introduced) are shown in FIG. 5. In the figure, an open square represents an RNA chain and a filled square represents a DNA chain. As is apparent from the figure, in polynucleotides of any base sequences, the expression of the gene introduced into CHO-KI cells was inhibited by the double-stranded polynucleotides of 21 nucleotides each in which at least about an upstream half region of the polynucleotide was RNA (FIGS. 5 (A), (C) and (E)).

The double-stranded polynucleotides of 21 nucleotides each prepared by identifying or predicting a domain necessary and sufficient for exhibition of the RNAi effect according to the method as used in the present Example and the other technique, reserving a part containing this domain as RNA, and substituting the other part with DNA are considered to be able to inhibit the expression of the target gene by the RNAi effect.

While the invention has been described in detail and with reference to specific examples thereof, it will be apparent to one skilled in the art that various changes and modifications can be made therein without departing from the spirit and scope thereof.

The present application is based on Japanese Patent Application No. 2001-355896, filed Nov. 21, 2001, the content of which is incorporated herein by reference.

### INDUSTRIAL APPLICABILITY

The present invention provides a means for directly analyzing functions of genes in human and other organisms, when a cultured cell, tissue, or individual organism is used. Moreover, according to the method of using an indicator, cells exhibiting an especially high RNAi effect can be primarily screened, so that an effective analysis can be achieved, even among cells exhibiting a weak RNAi effect.

As a conventional inventive technology for use in the same purpose, an RNAi method by introduction with a double-stranded RNA may be mentioned but there are problems in that RNA is extremely easily degraded by a ribonuclease, especially in a single-stranded state and cost of the production is expensive. In the present invention, the polynucleotide to be introduced is not limited to be entirely RNA. Therefore, use of a polynucleotide comprising DNA and RNA, specifically, a hybrid polynucleotide of a DNA strand and an RNA strand or a DNA-RNA chimera polynucleotide, enables enhancement of stability of the polynucleotide, as a substance, for the introduction and also reduction of the production cost. Therefore, according to the present invention, it is possible to develop the polynucleotide itself as a pharmaceutical preparation for treating diseases. Moreover, modifications such as fluorescent labeling, biotin labeling, amination, phosphorylation, and thiolation can be easily conducted over a wide variety with respect to DNA, as compared with the case of RNA. Therefore, when it is used as a pharmaceutical agent or reagent, a function suitable for a purpose can be imparted by conducting such modifications.

Numerous modification and variations on the present invention are possible in light of the above teachings. It is therefore to be understood that within the scope of the appended claims, the invention may be practiced otherwise than as specifically described herein.

1. A method for inhibiting expression of a target gene, which comprises introducing into a cell, tissue, or individual organism a double-stranded polynucleotide comprising DNA and RNA having a substantially identical polynucleotide sequence with at least a partial nucleotide sequence of the target gene.

2. The method according to claim 1, wherein the double-stranded polynucleotide comprises a self complementary single strand.

3. The method according to claim 1, wherein the double-stranded polynucleotide is a hybrid of a DNA strand and an RNA strand.

4. The method according to claim 3, wherein the hybrid of a DNA strand and an RNA strand comprises a sense strand DNA and an antisense strand RNA.

5. The method according to claim 1, wherein the double-stranded polynucleotide is a chimera of DNA and RNA.

6. The method according to claim 1, wherein, in the double-stranded polynucleotide, at least an upstream partial region of the polynucleotide is RNA.

7. The method according to claim 6, wherein the upstream partial region consists of 9 to 13 nucleotides.

8. The method according to claim 1, wherein the double-stranded polynucleotide consists of 19 to 25 nucleotides and at least an upstream half region of the polynucleotide is RNA.

9. The method according to claim 1, wherein the target gene is plural.

10. A method of analyzing a function of a gene, which comprises analyzing a phenotypic change appearing in the cell, tissue, or individual organism as a result of inhibition of expression of a target gene by the method according to claim 1.

11. A method of imparting a specific property to a cell, tissue, or individual organism which comprises inhibiting expression of a target gene using the method according to claim 1.

12. A cell, tissue, or individual organism obtainable by the method according to claim 11.

13. A method of screening an agent for preventing and/or treating a disease associated with a target gene, which comprises adding a test substance to the cell, tissue, or individual organism according to claim 12 and analyzing a property change imparted to the cell, tissue, or individual organism.

14. A method for treating a disease associated with a target gene, comprising administering a substance obtainable by the method according to claim 13 to a patient in need of such treatment.

15. A method of producing a preventing and/or therapeutic agent for a disease associated with a target gene, which comprises pharmaceutically formulating a substance selected by the method according to claim 13.

16. The method according to claim 1, which further comprises introducing into the cell, tissue, or individual organism an expression vector comprising DNA encoding an indicator protein, and subsequently selecting and analyzing the cell, tissue, or individual organism having a quantity of a signal generated from the indicator protein of a specific strength or greater.

17. The method according to claim 1, which further comprises introducing into the cell, tissue, or individual organism an expression vector comprising DNA encoding an indicator protein and a double-stranded RNA comprising a substantially identical nucleotide sequence with at least a partial nucleotide sequence of the DNA and subsequently selecting and analyzing the cell, tissue, or individual organism having a reduced quantity of a signal generated from the indicator protein.

18. The method according to claim 16, wherein the indicator protein is a protein in which the quantity of the protein and the quantity of a signal generated from the protein change in proportion.

19. The method according to claim 16, wherein the indicator protein is luciferase.

20. A method of identifying a functional domain of RNA in an RNAi method, which comprises (i) preparing a double-stranded polynucleotide having a substantially identical nucleotide sequence with at least a partial nucleotide sequence of a target gene and comprises a chimera of DNA and RNA, (ii) introducing into a cell, tissue, or individual organism the double-stranded polynucleotide, (iii) measuring an inhibition degree of expression of the target gene in the cell, tissue, or individual organism, and (iv) identifying a sequence which is RNA for inhibiting expression of the target gene.

21. The method according to claim 20, wherein either of the double strands of the double-stranded polynucleotide is an RNA strand.

22. A double-stranded polynucleotide for inhibiting expression of a target gene according to the method of claim 1.

23. A method for treating a disease associated with a target gene, comprising administering a substance comprising at least the double-stranded polynucleotide according to claim 22 to a patient in need of such treatment.

24. A kit for conducting the method according to claim 1, comprising at least a double-stranded polynucleotide comprising DNA and RNA having a substantially identical polynucleotide sequence with at least a partial nucleotide sequence of a target gene of a patient in need of such treatment.

25. A double-stranded polynucleotide for identifying a functional domain of RNA according to the method of claim 20.

26. A kit for conducting the method according to claim 20, comprising at least a double-stranded polynucleotide comprising DNA and RNA having a substantially identical polynucleotide sequence with at least a partial nucleotide sequence of a target gene of a patient in need of such treatment.
